SEQUENCE LISTING (2 sequences)

SEQ ID NO: 1 - protein, 449 aa, Artificial
  Synthetic NKTT320 Heavy chain sequence
EVQLVESGGGLVQPGGSLRLSCVASGFTFSNYWMNWVRQAPGKGLEWVAEIRLKSNNYATHYAESVKGRFTISRDDSKNTVYLQMNSLRAEDTAVYYCTRNGNYVDYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial
  Synthetic NKTT320 Light chain sequence
DIQMTQSPSSLSASVGDRVTITCKASQDVSTAVAWYQQKPGQAPRLLIYWASTRHTGVPSRFSGSGSGTDFTLTISSLQPEDFALYYCQQHYSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2017/0182077 A1
(43) Pub. Date: Jun. 29, 2017

(54) METHODS AND PHARMACEUTICAL COMPOSITIONS FOR THE TREATMENT OF ACUTE EXACERBATIONS OF CHRONIC OBSTRUCTIVE PULMONARY DISEASE

(71) Applicants: INSERM (INSTITUT NATIONAL DE LA SANTÉ ET DE LA RECHERCHE MÉDICALE), PARIS (FR); INSTITUT PASTEUR DE LILLE, Lille (FR); CENTRE NATIONAL DE LA RECHERCHE SCIENTIFIQUE (CNRS), PARIS (FR); UNIVERSITÉ DE LILLE 1 SCIENCES ET TECHNOLOGIES, Villeneuve d'Ascq (FR); UNIVERSITÉ DE DROIT ET DE LA SANTÉ DE LILLE 2, Lille (FR)

(72) Inventors: Philippe GOSSET, Lille Cedex (FR); Muriel PICHAVANT, Lille Cedex (FR); Gaëlle REMY, Lille Cedex (FR)

(21) Appl. No.: 15/118,260

(22) PCT Filed: Feb. 13, 2015
(86) PCT No.: PCT/EP2015/053041

§ 371 Date: Aug. 11, 2016

(30) Foreign Application Priority Data
Feb. 14, 2014 (EP) 14305201.7

(51) Int. Cl. A61K 31/7032; A61K 39/09; A61K 39/102; C07K 16/28; A61K 45/06

(52) U.S. Cl. CPC A61K 31/7032; C07K 16/2809; A61K 45/06; A61K 39/102; A61K 39/092; A61K 2039/505

(57) ABSTRACT

The present invention relates to methods and pharmaceutical compositions for the treatment of acute exacerbation of chronic obstructive pulmonary disease. In particular, the present invention relates to a method of treating acute exacerbation of chronic obstructive pulmonary disease in a subject in need thereof comprising administering the subject with a therapeutically effective amount of at least one NKT cell agonist.

### FIELD OF THE INVENTION

The present invention relates to methods and pharmaceutical compositions for the treatment of acute exacerbation of chronic obstructive pulmonary disease.

### BACKGROUND OF THE INVENTION

Chronic obstructive pulmonary disease (COPD) represents a severe and increasing global health problem. By 2020, COPD will have increased from 6th (as it is currently) to the 3rd most common cause of death worldwide. In the United States, COPD is believed to account for up to 120,000 deaths per year. The clinical course of COPD is characterized by chronic disability, with intermittent, acute exacerbations which may be triggered by a variety of stimuli including exposure to pathogens, inhaled irritants (e.g., cigarette smoke), allergens, or pollutants. “Acute exacerbation” refers to worsening of a subject's COPD symptoms from his or her usual state that is beyond normal day-to-day variations, and is acute in onset. Acute exacerbations of COPD greatly affect the health and quality of life of subjects with COPD. Acute exacerbation of COPD is a key driver of the associated substantial socioeconomic costs of the disease. Multiple studies have also shown that prior exacerbation is an independent risk factor for future hospitalization for COPD. In conclusion, exacerbations of COPD are of major importance in terms of their prolonged detrimental effect on subjects, the acceleration in disease progression and the high healthcare costs. However up to now there is no method for the treatment of acute exacerbation of COPD.

### SUMMARY OF THE INVENTION

The present invention relates to methods and pharmaceutical compositions for the treatment of acute exacerbation of chronic obstructive pulmonary disease. In particular, the present invention is defined by the claims.

### DETAILED DESCRIPTION OF THE INVENTION

The present invention relates to a method of treating acute exacerbation of chronic obstructive pulmonary disease in a subject in need thereof comprising administering the subject with a therapeutically effective amount of at least one NKT cell agonist.

As used herein the term “acute exacerbation” has its general meaning in the art and refers to worsening of a subject's COPD symptoms from his or her usual state that is beyond normal day-to-day variations, and is acute in onset. Typically, the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, and said method comprises reducing the frequency, severity or duration of one or more of said symptoms. Acute exacerbation may have various etiologies, but typically may be caused by viral infections, bacterial infections, or air pollution. For example, approximately 50% of acute exacerbations are due primarily to the bacteria Streptococcus pneumoniae (causing pneumonia), Haemophilus influenzae (causing flu), and Moraxella catarrhalis (causing pneumonia). Viral pathogens associated with acute exacerbations in subjects with COPD include rhinoviruses, influenza, parainfluenza, coronavirus, adenovirus, and respiratory syncytial virus.

In some embodiments, the acute exacerbation of COPD is caused by a bacterial infection. In some embodiments, the acute exacerbation of COPD is caused by a viral infection. In some embodiments, the acute exacerbation of COPD is caused by air pollution.

In some embodiments, the subject experienced an acute exacerbation of COPD or is at risk of experiencing an acute exacerbation of COPD. In some embodiments, the subject has experienced at least one acute exacerbation of COPD in the past 24 months. In one particular embodiment, the subject has experienced at least one acute exacerbation of COPD in the past 12 months. In some embodiments, subject is a frequent exacerbator. As used herein the term “frequent exacerbator” refers to a subject who suffers from or is undergoing treatment for COPD and who experiences at least 2, and more typically 3 or more, acute exacerbations during a 12 month period.

In some embodiments of the present invention, “treating” refers to treating an acute exacerbation of COPD, reducing the frequency, duration or severity of an acute exacerbation of COPD, treating one or more symptoms of acute exacerbation of COPD, reducing the frequency, duration or severity of one or more symptoms of an acute exacerbation of COPD, preventing the incidence of acute exacerbation of COPD, or preventing the incidence of one or more symptoms of acute exacerbation of COPD, in a human. The reduction in frequency, duration or severity is relative to the frequency, duration or seventy of an acute exacerbation or symptom in the same human not undergoing treatment according to the methods of the present invention. A reduction in frequency, duration or severity of acute exacerbation or one or more symptoms of acute exacerbation may be measured by clinical observation by an ordinarily skilled clinician with experience of treating COPD subjects or by subjective self evaluations by the subject undergoing treatment. Clinical observations by an ordinarily skilled clinician may include objective measures of lung function, as well as the frequency with which intervention is required to maintain the subject in his or her most stable condition, and the frequency of hospital admission and length of hospital stay required to maintain the subject in his or her most stable condition. Typically, subjective self evaluations by a subject are collected using industry-recognized and/or FDA-recognized subject reported outcome (PRO) tools. Such tools may allow the subject to evaluate specific symptoms or other subjective measures of quality of life. An example of one subject reported outcome tool is Exacerbations from Pulmonary Disease Tool (EXACT-PRO), which is currently being developed for evaluating clinical response in acute bacterial exacerbations by United BioSource Corporation along with a consortium of pharmaceutical industry sponsors in consultation with the FDA.

In some embodiments, the treatment is a prophylactic treatment. As used herein, the term “prophylactic treatment” refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

As used herein the term “NKT cell” has its general meaning in the art and refers to Natural killer T cells (NKT cells). Typically, natural killer T cells (NKT cells) specifically recognize self lipid-based or foreign lipid-based antigens bound to the major histocompatibility complex (MHC) class I homolog CD1d. NKT cells can be divided into 2 main subsets: Type 1 which express an invariant T cell receptor and are CD1d-restricted (iNKT), Type 2 (NKT) which express varied T cell receptors, but are CD1d-restricted.

As used herein, the term “NKT cell agonist” refers to any compound natural or not which has the ability to stimulate NKT cells. Typically the activation is assayed by the production of cytokines. In particular, NKT cells are activated when they produced interferon gamma, IL 4, IL10, IL 13 or IL22. More particularly, a compound is considered as a NKT cell agonist when the compound is able to induce production of IL22 by NKT cells.

In some embodiments, the NKT cell agonist includes any derivative or analogue derived from a lipid, that is typically presented in a CD1d context by antigen presenting cells (APCs) and that can promote, in a specific manner, cytokine production by NKT cells. Typically the NKT cell agonist is a alpha-galactosylceramide compound.

As used herein, the term “alpha-galactosylceramide compound” or “alpha-GalCer compound” has its general meaning in the art and refers to any derivative or analogue derived from a glycosphingolipid that contains a galactose carbohydrate attached by an α-linkage to a ceramide lipid that has an acyl and sphingosine chains of variable lengths (Van Kaer L. α-Galactosylceramide therapy for autoimmune diseases: Prospects and obstacles. Nat. Rev. Immunol. 2005; 5: 31-42).

Various publications have described alpha-galactosylceramide compounds and their synthesis. An exemplary, but by no means exhaustive, list of such references includes Morita, et al., J. Med. Chem., 25 38:2176 (1995); Sakai, at al., J. Med. Chem., 38:1836 (1995); Morita, et al., Bioorg. Med. Chem. Lett., 5:699 (1995); Takakawa, etal., Tetrahedron, 54:3150 (1998); Sakai, at al., Org. Lett., 1:359 (1998); Figueroa-Perez, et al., Carbohydr. Res., 328:95 (2000); Plettenburg, at al., J. Org. Chem., 67:4559 (2002); Yang, at al., Angew. Chem., 116:3906 (2004); Yang, at al., Angew. Chem. Int. Ed., 43:3818 (2004); and, Yu, etal., Proc. Natl. Acad. Sci. USA, 102(9):3383-3388 (2005).

Examples of patents and patent applications describing instances of α-galactosylceramide compounds include U.S. Pat. No. 5,936,076; U.S. Pat. No. 6,531,453 U.S. Pat. No. 5,S53,737, U.S. Pat. No. 8,022,043, US Patent Application 2003030611, US Patent Application 20030157135, US Patent Application 20040242499, US Patent Application 20040127429, US Patent Application 20100104590, European Patent EP0609437 and International patent application W02006026389.

A typical alpha-galactosylceramide compound is KRN7000 ((2S 3S, 4R)-1-0-(alfaD-galactopyranosyl)-N-hexacosanoyl-2-amino-1,3,4-octadecanetriol)) (KRN7000, a novel immunomodulator, and its antitumor activities. Kobayashi E, Motoki K, Uchida T, Fukushima H, Koezuka Y. Oncol Res. 1995; 7(10-11):529-34.).

Other examples includes:

(2S,3R)-1-(a-D-galactopyranosyloxy)-2-tetracosanoylamino-3-octadecanol,

(2S,3 R)-2-docosanoylamina-1-(a-Dgalactopyranosyloxy)-3-octadecanol,

(2S,3R)-1-(a-D-galactopyranosyloxy)-2-icosanoylamino-3-octadecanol,

(2S,3R)-1-(a-D-galactopyranosyloxy)-2-octadecanoylamino-3-octadecanol,

(2S,3R)-1-(a-D-galactopyranosyloxy)-2-tetradecanoylamino-3-octadecanol,

(2S,3R)-2-decanoylamino-1-(a-D-40galactopyranosyloxy)-3-octadecanol,

(2S,3R)-1-(a-D-galactopyranosyloxy)-2-tetracosanoylamino-3-tetradecanol,

(2S,3R)-1-(a-D-galactopyranosyloxy)-2-tetradecanoylamino-3-hexadecanol,

(2R,3S)-1-(a-D-galactopyranosyloxy)-2-tetradecanoylamino-3-hexadecanol,

(2S,3S)-1-(a-D-galactopyranosyloxy)-2-tetradecanoylamino-3-hexadecanol,

(2S,3R)-1-(a-D-galactopyranosyloxy)-2[(R)-2-hydroxytetracosanoylamino]-3-octadecanol,

(2S,3R,4E)-1-(a-D-galactopyranosyloxy)-2-octadecanoylamino-4-octadecen-3-ol,

(2S,3R,4E)-1-(a-D-galactopyranosyloxy)-2-tetradecanoylamino-4-octadecen-3-ol,

(2S,3S,4R)-1-(a-D-galactopyranosyloxy)-2-tetracosanoylamino-3,4-octadecanediol,

(2S,3S,4R)-1-(a-D-galactopyranosyloxy)-2-tetracosanoylamino-3,4-heptadecanediol,

(2S,3S,4R)-1-(a-D-galactopyranosyloxy)-2-tetracosanoylamino-3,4-pentadecanediol,

(2S,3S,4R)-1-(a-D-galactopyranosyloxy)-2-tetracosanoylamino-3,4-undecanediol,

(2S,3S,4R)-1-(a-D-galactopyranosyloxy)-2-hexacosanoylamino-3,4-heptadecanediol,

(2S,3S,4R)-1-(a-D-galactopyranosyloxy)-2-[(R)-2-hydroxytetracosanoylamino]-3,4-octadecanediol,

(2S,3S,4R)-1-(a-D-galactopyranosyloxy)-2-[(R)-2-hydroxytetracosanoylamino]-3,4-heptadecanediol,

(2S,3S,4R)-1-(a-D-galactopyranosyloxy)-2-[(R)-2-hydroxytetracosanoylamino]-3,4-pentadecanediol,

(2S,3S,4R)-1-(a-D-galactopyranosyloxy)-2-[(R)-2-hydroxytetracosanoylamino]-3,4-undecanediol,

(2S,3S,4R)-1-(a-D-galactopyranosyloxy)-2-[(R)-2-hydroxyhexacosanoylamino]-3,4-octadecanediol,

(2S,3S,4R)-1-(a-D-galactopyranosyloxy)-2-[(R)-2-hydroxyhexacosanoylamino]-3,4-nonadecanediol,

(2S,3S,4R)-1-(a-D-galactopyranosyloxy)-2-[(R)-2-hydroxyhexacosanoylamina]-3,4-icosanediol,

(2S,3S,4R)-1-(a-D-galactopyranosyloxy)-2-[(S)-2-hydroxytetracosanoylamino]-3,4-heptadecanediol,

(2S,3S,4R)-1-(a-D-galactopyranosyloxy)-2-[(R)-2-hydroxytetracosanoylamino]-3,4-hexadecanediol,

(2S,3S,4R)-1-(a-D-galactopyranosyloxy)-2-[(S)-2-hydroxytetracosanoylamino]-16-methyl-3,4-heptadecanediol,

(2S,3S,4R)-1-(a-D-galactopyranosyloxy)-16-methyl-2-tetracosanoylamino-3,4-heptadecanediol,

(2S,3S,4R)-1-(a-D-galactopyranosyloxy)-2-[(R)-2-hydroxytricosanoylamino]-16-methyl-3,4-heptadecanediol,

(2S,3S,4R)-1-(a-D-galactopyranosyloxy)-2-[(R)-2-hydroxypentacosanoylamino]-16-methyl-3,4-octadecanediol,

(2S,3R)-1-(a-D-galactopyranosyloxy)-2-oleoylamino-3-octadecanol,

(2S,3S,4R)-1-(a-D-galactopyranosyloxy)-2-hexacosanoylamino-3,4-octadecanediol;

(2S,3S,4R)-1-(a-D-galactopyranosyloxy)-2-octacosanoylamino-3,4-heptadecanediol

(2R,3R)-1-(a-D-galactopyranosyloxy)-2-tetradecanoylamino-3-hexadecanol

(2S,3R,4S,5R)-2-((2S,3S,4R)-2-(4-hexyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-2-(4-heptyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-2-(4-hexadecyl-1H-1,2,3-triazol-1-yl)-3,4-dihydroxyoctadecyloxy)-6-(hydroxymethyl)-tetrahydro-28-pyrane-3,4,5-triol;

(2S,3R,4S,5R)-2-((2S,3S,4R)-3,4-dihydroxy-2-(4-tricosyl-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-tetracosyl-1H-1,2,3-triazol-1-yl)octadecyloxy)-6-(hydroxymethyl)-tetrahydro-2H-pyrane-3,4,5-triol;

(2S,3R,4S,5R)-2-((2S,3S,4R)-3,4-dihydroxy-2-(4-pentacosyl-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(6-phenylhexyl)-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(7-phenylheptyl)-1H-1,2,3-triazol-1-yl)octadecyloxy)-6-(hydroxymethyl)-tetrahydro-28-pyrane-3,4,5-triol;

(2S,3R,4S,5R)-2-((2S,3S,4R)-3,4-dihydroxy-2-(4-(8-phenyloctyl)-1H-1,2,3-triazol-1-yl)octadecyloxy)-6-(hydroxymethyl)-tetrahydro-28-pyrane-3,4,5-triol;

11-amino-N-((2S,3S,4R)-3,4-dihydroxy-1-((2S,3R,4S,5R)-3,4,5-trihydroxy-6-(hydroxymethyl)-tetrahydro-28-pyran-2-yloxy)octadecan-2-yl)undecanamide;

12-amino-N-((2S,3S,4R)-3,4-dihydroxy-1-((2S,3R,4S,5R)-3,4,5-trihydroxy-6-(hydroxymethyl)-tetrahydro-2H-pyran-2-oxy)octadecan-2-yl)dodecanamide;

N-((2S,3S,4R)-3,4-dihydroxy-1-((2S,3R,4S,5R)-3,4,5-trihydroxy-6-(hydroxymethyl)-tetrahydro-2Hpyran-2-yloxy)octadecan-2-yl)-11-hydroxyundecanamide;

N-((2S,3S,4R)-3,4-dihydroxy-1-((2S,3R,4S,5R)-3,4,5-trihydroxy-6-(hydroxymethyl)-tetrahydro-2Hpyran-2-yloxy)octadecan-2-yl)-12-hydroxydodecanamide;

8-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)octanamide;

N-((2S,3S,4R)-3,4-dihydroxy-1-((2S,3R,4S,5R)-3,4,5-trihydroxy-6-(hydroxymethyl)-tetrahydro-2Hpyran-2-yloxy)octadecan-2-yl)-11-(dipentylamino)undecanamide;

11-(diheptylamino)-N-((2S,3S,4R)-3,4-dihydroxy-1-((2S,3R,4S,5R)-3,4,5-trihydroxy-6-(hydroxymethyl)-tetrahydro-2H-pyran-2-yloxy)octadecan-2-yl)undecanamide;

N-((2S,3S,4R)-3,4-dihydroxy-1-((2S,3R,4S,5R)-3,4,5-trihydroxy-6-(hydroxymethyl)-tetranydro-2Hpyran-2-yloxy)octadecan-2-yl)-11-mercaptoundecanamide;

N-((2S,3S,4R)-3,4-dihydroxy-1-((2S,3R,4S,5R)-3,4,5-dihydroxy-6-(hydroxymethyl)-tetrahydro-2Hpyran-2-yloxy)octadecan-2-yl)-12-mercaptododecanamide,

In some embodiments alpha-galactosylceramide compounds are pegylated. As used herein, the term “pegylated” refers to the conjugation of a compound moiety (i.e. α-galactosylceramide compound) with conjugate moiety(ies) containing at least one polyalkylene unit. In particular, the term pegylated refers to the conjugation of the compound moiety (i.e. alpha-galactosylceramide compound) with a conjugate moiety having at least one polyethylene glycol unit.

In some embodiments, the NKT cell agonist of the present invention consists in a particulate entity comprising at least one alpha-galactosylceramide compound and at least one targeting agent that targets in vivo said to alpha-galactosylceramide compound to human BDCA3+ dendritic cells.

In some embodiments, said targeting agent is a molecule that specifically binds to a cell surface marker of human BDCA3+ dendritic cells. In some embodiments, a “cell surface marker” of human BDCA3+ dendritic cells refers to a protein or a biomolecule of human BDCA3+ dendritic cells, that is expressed on the external surface of BDCA3+ cells. More specifically, it may correspond to an antigenic determinant of BDCA3+ cells that is expressed on the surface of BDCA3+ dendritic cells and can be recognized specifically by antibodies. Preferably, the targeting agent binds to a cell surface marker that is specific of BDCA3+ cells, i.e. that is not expressed on other dendritic cells (or at a lower level). Typically, BDCA3+ dendritic cells are Lin-(CD3,C14,CD16, CD19, CD20, CD56), HLA-DR+, BDCA3+ (also known as CD141), Clec9A+, XCR-1+, TLR3+, CD11c+. Accordingly, in one specific embodiment, said targeting agent is a binding molecule to a cell surface marker of BDCA-3+ dendritic cells selected from the group consisting of CLEC9A or XCR-1. Accordingly, in some embodiments, the particulate entity comprises, as a targeting agent, a molecule that binds specifically to CLEC9A and/or to XCR-1, typically, to the extracellular domain of CLEC9A or to the extracellular domain of XCR-1.

Any molecule known to have binding specificity towards a cell surface marker of human dendritic cells, preferably towards human BDCA3+ specific cell surface marker, can be used for preparing the particulate entity of the invention. Antibodies are particularly appropriate since antibodies with desired binding specificity may be routinely generated, for example by screening antibody libraries against the desired target. Screening methods may include for example, phage display technologies or other related technologies known in the Art. Such antibodies may also be easily grafted to nanoparticles or directly conjugated to the the αGalCer compound, using conventional chemical coupling technologies.

Ideally, the nanoparticle may have the following features: it is biocompatible, and it can physically couple the alpha-galactosylceramide compound and the targeting agent via covalent or non-covalent linkage.

“Physical coupling” may result from either covalent binding of the targeting agent and/or αGalCer compound to a constituent of the nanoparticle or via non-covalent, such as electrostatic or ionic interactions.

Any nanoparticles which have been described in the art for in vivo delivery of active principles in human may be used. Such nanoparticles include for example liposomes and micelles, nanosphere or nanoparticles, nanotubes, nanocrystals, hydrogels, carbon-based nanoparticles and the like (see for example Peer et al., 2007, Nature nanotechnology, vol. 2, pp751-760). Examples of suitable nanoparticles are also described for example in Cruz et al J Control Release 2010,144(2):118-26.

Typically, the nanoparticle according to the invention has a mean diameter between 1 to 2000 nm diameter, for example between 10 to 500 nm or between 10 to 200 nm. As used herein, the size of a nanoparticle may correspond to the mean value±SD of ten readings from dynamic light scattering measurements as described in Cruz et al, 2011, Cruz et al., 2010^{30,31}.

The nanoparticles of the invention may comprise an inorganic core, such as, but not limited to, semiconductor, metal (e.g. gold, silver, copper, titanium, nickel, platinum, palladium and alloys), metal oxide nanoparticles (e.g. Cr2O3, Co3O4, NiO, MnO, CoFe2O4, and MnFeO4).

In other embodiments, the nanoparticles comprises at least a core with one or more polymers, or their copolymer, such as, e.g., one or more of dextran, carboxymethyl dextran, chitosan, trimetylchitosan, polyvinylalcohol (PVA), polyanhydrides, polyacylates, polymethacrylates, polyacylamides, cellulose, hydromellose, starch, dendrimers, polyamino acids, polyethyleneglycols, polyethyleneglycol-co-propyleneglycol, aliphatic polyesters, including poly(lactic acid (PLA), poly(glycolic acid), and their copolymers including poly(lactic-co-glycolylic)acid (PLGA), or poly(ε-caprolactone).

In general the surface of the nanoparticles may also be functionalised or coated to produce a desirable physical characteristic such as solubility, biocompatibility, and for facilitating chemical linkages with other biomolecules, such as the α-galactosylceramide or the targeting agent.

For example, the surface of the nanoparticles can be functionalized by incorporating one or more chemical linkers such as, without limitation: carboxyl groups, amine groups, carboxyl/amine, hydroxyl groups, polymers such as silane, dextran or PEG or their derivatives.

In a specific embodiment, nanoparticle has a core that comprises polymers selected from the group consisting of: poly(lactic acid), poly(glycolic acid), or mixtures thereof. In another specific embodiment, the nanoparticle comprise poly(lactic)poly(glycolic) acid co-polymers (PLGA). Other suitable polymers may comprise polyamino acid selected from the group consisting of poly(g-glutamic acid), poly(a-aspartic acid), poly(e-lysine), poly(a-glutamic acid), poly(a-lysine), poly-asparagine, or derivatives thereof, and mixtures thereof.

In a specific embodiment, the nanoparticles of the invention comprise a core containing polymers and a coating, and the targeting agent is attached to the nanoparticle by covalent linkage to the surface of the coating. In a further specific embodiment, the nanoparticles comprises:
a core made of poly(lactic acid), poly(glycolic acid), or their copolymers, with a coating on its surface,
an efficient amount of the alpha-galactosylceramide compound,
antibody covalently attached to the coating of the nanoparticle, wherein said antibody binds specifically to BDCA3+ dendritic cells.

Other suitable nanoparticles include oxide and hybrid nanostructures such as iron oxide nanoparticle or polymer-based nanoparticle, optionally coated with organic or inorganic stabilizers, such as silane, dextran or PEG (see e.g. S. Chandra et al./Advanced Drug Delivery Rev (2011), doi:10.1016/j.adr.2011.06.003).

Methods for encapsulating or chemically coupling the α-galactosylceramide compound, such as αGalCer compound, and/or the targeting agent to the nanoparticles are known in the art. For example, the nanoparticle is prepared together with αGalCer compound, and the αGalCer is encapsulated (retained by non-covalent binding) into the nanoparticle. Alternatively, the nanoparticle is prepared and the the α-galactosylceramide compound, is chemically linked to the functionalized surface of the nanoparticle, via conventional coupling techniques. Example of preparation of PLGA based nanoparticles, with encapsulated αGalCer is described in Cruz et al, 2011 [Mol Pharm 2011, 8:520-531], and Cruz et al. 2010 [J Control Release 2010, 144:118-126].

In one specific embodiment, the nanoparticle comprises encapsulated αGalCer at amounts comprised between 0.01 and 1000 ng per mg of nanoparticle. In a specific embodiment, 1 ng to 1000 ng of the alpha-galactosylceramide compound per mg of nanoparticles is used. In a specific embodiment, the nanoparticle of the invention further comprises an antigenic determinant as described more in detail in the next sections. Such antigenic determinant may be encapsulated or attached to the surface of the nanoparticle, similarly to the targeting agent.

In some embodiments, the NKT cell agonist is an antibody. As used herein, “antibody” includes both naturally occurring and non-naturally occurring antibodies. Specifically, “antibody” includes polyclonal and monoclonal antibodies, and monovalent and divalent fragments thereof Furthermore, “antibody” includes chimeric antibodies, wholly synthetic antibodies, single chain antibodies, and fragments thereof The antibody may be a human or non human antibody. A non human antibody may be humanized by recombinant methods to reduce its immunogenicity in man.

In some embodiments of the antibodies or portions thereof described herein, the antibody is a monoclonal antibody. In some embodiments of the antibodies or portions thereof described herein, the antibody is a polyclonal antibody. In some embodiments of the antibodies or portions thereof described herein, the antibody is a humanized antibody. In some embodiments of the antibodies or portions thereof described herein, the antibody is a chimeric antibody. In some embodiments of the antibodies or portions thereof described herein, the portion of the antibody comprises a light chain of the antibody. In some embodiments of the antibodies or portions thereof described herein, the portion of the antibody comprises a heavy chain of the antibody. In some embodiments of the antibodies or portions thereof described herein, the portion of the antibody comprises a Fab portion of the antibody. In some embodiments of the antibodies or portions thereof described herein, the portion of the antibody comprises a F(ab′)2 portion of the antibody. In some embodiments of the antibodies or portions thereof described herein, the portion of the antibody comprises a Fc portion of the antibody. In some embodiments of the antibodies or portions thereof described herein, the portion of the antibody comprises a Fv portion of the antibody. In some embodiments of the antibodies or portions thereof described herein, the portion of the antibody comprises a variable domain of the antibody. In some embodiments of the antibodies or portions thereof described herein, the portion of the antibody comprises one or more CDR domains of the antibody.

Typically, antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the relevant antigenic forms. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant antigen (i.e. NKT antigen) may be provided by expression with recombinant cell lines, in particular in the form of human cells expressing the antigen (i.e. NKT antigen) at their surface. Recombinant forms of the antigen may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in some embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al.,/.Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the antibody is modified to reduce or inhibit the ability of the antibody to mediate antibody dependent cellular cytotoxicity (ADCC) and/or complement dependent cytotoxicity (CDC) functionality (i.e. an antibody with reduced Fc-effector function”). In particular, the antibodies of the present invention have no Fc portion or have an Fc portion that does not bind FcyRI and Clq. In some embodiments, the Fc portion of the antibody does not bind FcyRI, Clq, or FcyRIII. Antibodies with such functionality, in general, are known. There are native such antibodies, such as antibodies with an IgG4 Fc region. There also are antibodies with Fc portions genetically or chemically altered to eliminate the Antibody dependent cell cytotoxicity (ADCC) and/or complement dependent cytotoxicity (CDC) functionality.

In some embodiments, the antibody of the invention is the NKTT320 antibody as described in WO2013063395. In some embodiments, the antibody of the invention comprises a heavy chain having the amino acid sequence set forth as SEQ ID NO: 1. In some embodiments, the antibody of the invention comprises a light chain having the amino acid sequence set forth as SEQ ID NO: 2. In some embodiments, the antibody of the invention comprises a heavy chain having the amino acid sequence set forth as SEQ ID and comprises a light chain having the amino acid sequence set forth as SEQ ID NO: 2. In some embodiments, the antibody of the invention comprises the CDRs of the heavy chain having the amino acid sequence set forth as SEQ ID NO: 1. In some embodiments, the antibody of the invention comprises the CDRs of the light chain having the amino acid sequence set forth as SEQ ID NO: 2. In some embodiments, the antibody of the invention comprises the CDRs of the heavy chain having the amino acid sequence set forth as SEQ ID NO: 1 and the CDRs of the light chain having the amino acid sequence set forth as SEQ ID NO: 2.

| (NKTT320 Heavy chain sequence) | |
|---|---|
| SEQ ID NO: 1 | |
| EVQLVESGGG LVQPGGSLRL SCVASGFTFS NYWMNWVRQA PGKGLEWVAE IRLKSNNYAT | 60 |
| HYAESVKGRF TISRDDSKNT VYLQMNSLRA EDTAVYYCTR NGNYVDYAMD YWGQGTLVTV | 120 |
| SSASTKGPSV FPLAPCSRST SESTAALGCL VKDYFPEPVT VSWNSGALTS GVHTFPAVLQ | 180 |
| SSGLYSLSSV VTVPSSSLGT KTYTCNVDHK PSNTKVDKRV ESKYGPPCPP CPAPEFEGGP | 240 |
| SVFLFPPKPK DTLMISRTPE VTCVVVDVSQ EDPEVQFNWY VDGVEVHNAK TKPREEQFNS | 300 |
| TYRVVSVLTV LHQDWLNGKE YKCKVSNKGL PSSIEKTISK AKGQPREPQV YTLPPSQEEM | 360 |
| TKNQVSLTCL VKGFYPSDIA VEWESNGQPE NNYKTTPPVL DSDGSFFLYS RLTVDKSRWQ | 420 |
| EGNVFSCSVM HEALHNHYTQ KSLSLSLGK | 449 |
| (NKTT320 Light chain sequence) | |
| SEQ ID NO: 2 | |
| DIQMTQSPSS LSASVGDRVT ITCKASQDVS TAVAWYQQKP GQAPRLLIYW ASTRHTGVPS | 60 |
| RFSGSGSGTD FTLTISSLQP EDFALYYCQQ HYSTPWTFGQ GTKLEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | 214 |

By a “therapeutically effective amount” is meant a sufficient amount of a NKT cell agonist compound to treat acute exacerbation of chronic obstructive pulmonary disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the NKT cell agonist of the present invention is combined with pharmaceutically acceptable excipients, and optionally sustained-release matrices, such as biodegradable polymers, to form pharmaceutical compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

In some embodiments, the pharmaceutical composition of the present invention is administered to the respiratory tract. The respiratory tract includes the upper airways, including the oropharynx and larynx, followed by the lower airways, which include the trachea followed by bifurcations into the bronchi and bronchioli. Pulmonary delivery compositions can be delivered by inhalation by the subject of a dispersion so that the active ingredient within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a subject during administration of the aerosol medicament. Examples of pharmaceutical devices for aerosol delivery include metered dose inhalers (MDIs), dry powder inhalers (DPIs), and air-jet nebulizers.

In some embodiment, the NKT cell agonist of the present invention is administered to the subject in combination with an anti-bacterial agent, such as antibiotics or antiviral agents. Suitable antibiotics that could be coadministered in combination with the polypeptide include, but are not limited to, at least one antibiotic selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime, trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine. Example of antiviral agents include but are not limited to acyclovir, famciclovir, valaciclovir, ganciclovir, cidofovir; amantadine, rimantadine; ribavirin; zanamavir and/or oseltamavir; a protease inhibitor, such as indinavir, nelfinavir, ritonavir and/or saquinavir; a nucleoside reverse transcriptase inhibitor, such as didanosine, lamivudine, stavudine, zalcitabine, zidovudine; a non-nucleoside reverse transcriptase inhibitor, such as nevirapine, efavirenz.

Combination treatment may also include respiratory stimulants. Corticosteroids may be beneficial in acute exacerbations of COPD. Examples of corticosteroids that can be used in combination with the polypeptide (or the nucleic acid encoding thereof) are prednisolone, methylprednisolone, dexamethasone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate. Particularly preferred corticosteroids under the present invention are: dexamethasone, budesonide, beclomethasone, triamcinolone, mometasone, ciclesonide, fluticasone, flunisolide, dexamethasone sodium phosphate and esters thereof as well as 6α,9α-difluoro-17α-[(2-furanylcarbonyl)oxy]-11β-hydroxy-16α-methyl-3-oxoandrosta-1,4-diene-17β-carbothioic acid (S)-fluoromethyl ester. Still more preferred corticosteroids under the present invention are: budesonide, beclomethasone dipropionate, mometasone furoate, ciclesonide, triamcinolone, triamcinolone acetonide, triamcinolone hexaacetonide and fluticasone propionate optionally in the form of their racemates, their enantiomers, their diastereomers and mixtures thereof, and optionally their pharmacologically-compatible acid addition salts. Even more preferred are budesonide, beclomethasone dipropionate, mometasone furoate, ciclesonide and fluticasone propionate. The most preferred corticosteroids of the present invention are budesonide and beclomethasone dipropionate.

Bronchodilator dosages may be increased during acute exacerbations to decrease acute bronchospasm. Examples of bronchodilators include but are not limited to Iβ2-agonists (e.g. salbutamol, bitolterol mesylate, formoterol, isoproterenol, levalbuterol, metaproterenol, salmeterol, terbutaline, and fenoterol), anticholinergic (e.g. tiotropium or ipratropium), methylxanthined, and phosphodiesterase inhibitors.

In some embodiments, the NKT cell agonist of the invention is administered to the subject in combination with a vaccine which contains an antigen or antigenic composition capable of eliciting an immune response against a virus or a bacterium. Typically, the vaccine composition is used to eliciting an immune response against at least one bacterium selected from the group consisting of Streptococcus pneumoniae, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis. In particular, the vaccine composition is directed against Streptococcus pneumonia or Haemophilus influenza. More particularly, the vaccine composition is directed against Non-typeable Haemophilus influenzae (NTHi). Typically, vaccine composition typically contains whole killed or inactivated (eg., attenuated) bacteria isolate(s). However, soluble or particulate antigen comprising or consisting of outer cell membrane and/or surface antigens can be suitable as well, or instead of, whole killed organisms. In one or more embodiments, the outer cellular membrane fraction or membrane protein(s) of the selected isolate(s) is used. For instance, NTHi OMP P6 is a highly conserved 16-kDa lipoprotein (Nelson, 1988) which is a target of human bactericidal antibody and induces protection both in humans and in animal models. In chronic obstructive pulmonary disease (COPD), OMP P6 has been shown to evoke a lymphocyte proliferative response that is associated with relative protection from NTHi infection (Abe, 2002). Accordingly, OMP P6 or any other suitable outer membrane NTHi proteins, polypeptides (eg., P2, P4 and P26) or antigenic fragments of such proteins or polypeptides can find application for a NTHi vaccine. Soluble and/or particulate antigen can be prepared by disrupting killed or viable selected isolate(s). A fraction for use in the vaccine can then be prepared by centrifugation, filtration and/or other appropriate techniques known in the art. Any method which achieves the required level of cellular disruption can be employed including sonication or dissolution utilising appropriate surfactants and agitation, and combination of such techniques. When sonication is employed, the isolate can be subjected to a number of sonication steps in order to obtain the required degree of cellular disruption or generation of soluble and/or particulate matter of a specific size or size range. In some embodiments, the vaccine composition comprises an adjuvant, in a particular a TLR agonist. In some embodiments, the TLR agonist is selected from the group consisting of TLR1, TLR2, TLR3, TLR4, TLR5, TLR6, TLR7, TLR8, TLR9, TLR10, TLR11, TLR12, or TLR13 agonists.

In some embodiments, oxygen requirements may increase and supplemental oxygen may be provided.

The invention will be further illustrated by the following figures and examples. However, these examples and figures should not be interpreted in any way as limiting the scope of the present invention.

### FIGURES

FIG. 1—Pulmonary NKT cells. Air and COPD mice were intranasally challenged with Sp (5×10⁴ CFU/mouse). Lung tissues were collected 24 hours later, digested and processed to evaluate cellular inflammation. NKT cells were identified as CD45+ TCRβ+ PBS-57 loaded CD1d tetramer+ cells.

FIG. 2—Activation status of pulmonary NKT cells. Air and COPD mice were intranasally challenged with Sp (5×10⁴ CFU/mouse). Lung tissues were collected 24 hours later, digested and processed to evaluate cellular inflammation. CD69 expression was evaluated on CD45⁺ TCRβ⁺ PBS-57 loaded CD1d tetramer⁺ NKT cells. Results were expressed as mean±SEM of median of fluorescence intensity (MFI) (left panel). A representative histogram was reported in the right part for one mice of each group, the number representing the MFI for CD69 in each mice.

FIG. 3—Lung cells from Air and COPD mice were treated with αGC (100 ng/ml) or not (Ctl, non stimulated) for 48 hrs. The concentrations of IL-22, IL-17, IFN-γ and IL-4 were analyzed by ELISA in the supernatants. Values represented the mean±SEM.

FIG. 4—Lung mononuclear cells from Air and COPD mice were restimulated with PMA/ionomycin for 3 hours and analyzed for cytokine intracellular staining. Gated NKT cells (CD45+ TCRβ+ NK1.1+ cells) were analyzed for intra-cellular IL-17 and IL-22 production. Gates were set based on the relative isotype control. Mean of the percentage of positive cells (n=3, left histogram) as well as representative dot plots are shown.

FIG. 5—NKT cells from COPD patients have a defective cytokine response to S. pneumoniae.The percentage of positive cells for IL-17 and IL-22 was quantified by intracellular immuno-staining in mononuclear cells from healthy non-smoker subjects (n=14), healthy smokers (n=14) and COPD patients (n=12) (A). Percentages of IL-17 and IL-22 producing cells were measured by intracellular staining in NKT cells (CD3+, Vα24⁺ cells). Data represent mean±SEM. *: p<0.05 versus Medium in the different groups (one-way ANOVA test).

### EXAMPLE

Material & Methods:

Cigarette Smoke Exposure

C57B1/6 mice were exposed to CS generated from 5 cigarettes per day, 5 days a week, over a period of 12 weeks using a smoking machine (Emka, Scireq, Canada).

Bacterial Infection

Mice were inoculated with a clinical isolate of S. pneumoniae serotype 1 (Sp) (5×10⁴ cfu). Bacteria stocks were kept frozen at −80° C. Bacteria were thawn just before infection, and the number of cfu was checked on chocolate plates. Infection was performed by intranasal route (50 μl/mouse).

NKT Cell Characterization

Pulmonary cells from air or COPD mice were prepared as previously described and were analyzed by flow cytometry. NKT cells were identified as CD45⁺ TCRβ⁺ PBS57-loaded CD1d tetramer⁺ cells. Cell activation was estimated by flow cytometry using the expression of CD69 marker. To analyze NKT cell cytokine profile, pulmonary cell suspensions were incubated with phorbol 12-myristate 13-acetate (PMA; 20 ng/ml) and ionomycin (500 ng/ml) for 3 h. Cells were stained with PE-conjugated PBS57-loaded CD1d tetramer and FITC-conjugated TCRβ, and then fixed, permeabilized, and incubated with PE-conjugated mAb against IL-22 and APC-conjugated mAb against IL-17, or control rat IgG1 mAb in permeabilization buffer. Cells were acquired and analyzed on a Fortessa (Becton Dickinson, Rungis, France) cytometer, and using the FlowJo software respectively.

Cytokine production was analyzed in total lung cells. For this, 5×10⁵ lung cells were seeded on 96-well plates and then stimulated with alpha-GalactosylCeramide, or α-GC (100 ng/ml), and coated anti-CD3 Ab. Forty-eight hours later, supernatants were collected and analyzed for IFN-γ, IL-4, IL-17 and IL-22 concentration by ELISA (R&D Systems).

Patients with COPD

Peripheral blood and induced or spontaneous sputum were collected in stable COPD patients (n=10), in smokers (without COPD, n=13)) and in non smoker healthy controls (n=14) (CPP 2008-A00690-55) in order to evaluate ex vivo the Th17 response to infection with S. pneumoniae. Peripheral blood mononuclear cells (PBMC) were purified on Ficoll Paque gradient and 3×10⁶ cells/ml in complete RPMI1640 were exposed to S. pneumoniae (MOI=2) or to a positive control, phytohemagglutinin (1 μg/ml) (PHA, Difco). After 90 min, antibiotics were added to stop bacteria growth and supernatants were collected after 24 h incubation. In parallel, another batch of cells was incubated with brefeldin-A (10 μg/ml, Sigma) for 4 h before collection and was used for intracellular immuno-staining of cytokines in lymphocytes.

Results:

The Response of NKT Cells to Infection by S. pneumoniae is Altered in COPD Mice.

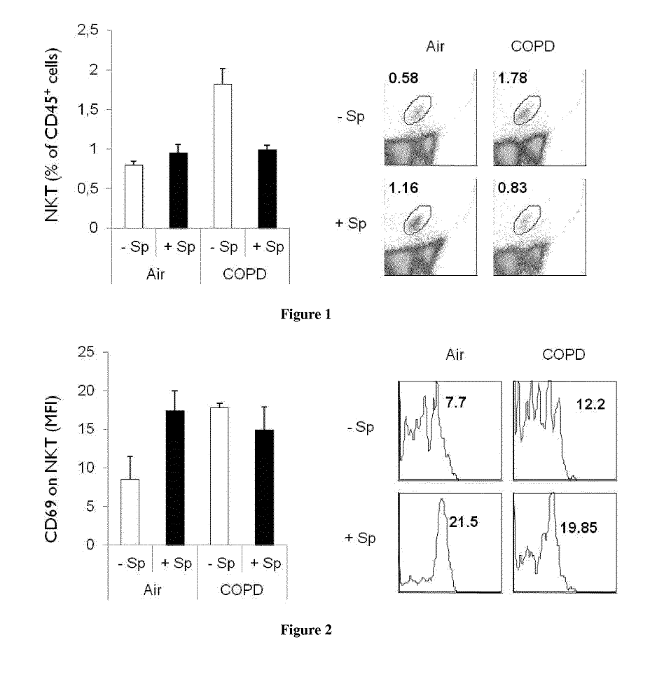

Repeated exposure of C57BL/6 mice to CS induced an inflammatory lung reaction. This was characterized by neutrophil, NK cell and macrophage recruitment (+30-50%) and/or activation after chronic exposure to CS, compared to mice exposed to ambient air. The frequency and number of pulmonary CD45⁺ TCRb⁺ PBS57-loaded CD1d tetramer⁺ invariant NKT cells was enhanced after CS exposure (FIG. 1). An increased expression of the activation marker CD69 on these NKT cells was also observed (FIG. 2). In response to Sp, Air mice showed a slightly higher recruitment of NKT cells, and these iNKT cells are strongly activated (FIGS. 1 and 2). In contrast, infection by Sp decreased the number of lung iNKT cells and the expression of CD69 was not increased in COPD mice.

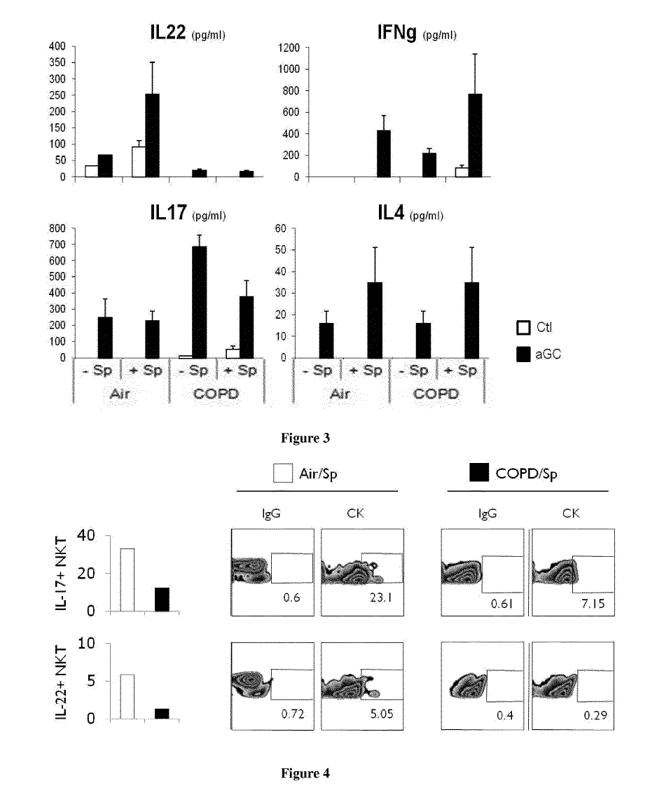

Stimulation of pulmonary cells from Air mice with the prototypical NKT cell activator alpha-GalactosylCeramide (aGC) resulted in a higher production of IL-22 after Sp challenge, but this was absent in COPD mice (FIG. 3). IL-17 levels were higher in non infected COPD mice compared to air mice. Challenge with Sp failed to increase IL-17 production in Air and COPD mice. Sp infection induced higher levels of IFN-y and IL-4 in both Air and COPD mice.

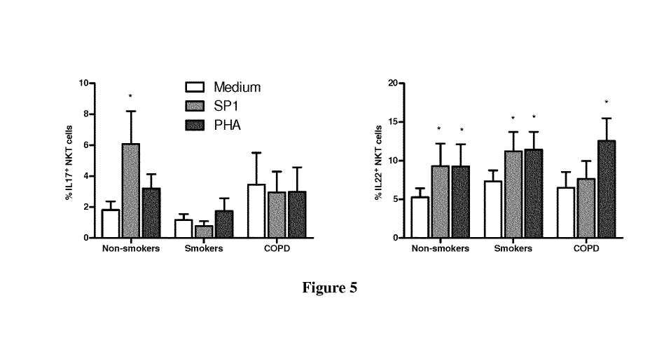

Cytokine production was also evaluated by flow-cytometry. Infection with Sp resulted in a higher frequency of IL-17⁺ and IL-22⁺ iNKT cells among lung cells from air-mice (at day 1 post infection). However, COPD mice showed a defect in the proportion of IL-17⁺ and IL-22⁺ NKT cells after Sp challenge (FIG. 4).These data demonstrated the NKT cells in the lung from Sp-infected COPD mice have an altered expression of CD69 and of the production of IL-17 and IL-22 in contrast with air-infected mice. Accordingly, stimulation of NKT cells by agonists is thus interesting for the treatment of acute exacerbation of chronic obstructive pulmonary disease.

Production of Th17 Cytokines in Response to S. pneumoniae is Altered in Peripheral Blood Mononuclear Cells from COPD Patients

In order to evaluate the production of Th17 cytokines in response to infection in COPD patients, their secretion was measured in the supernatants of mononuclear cells exposed to Streptococcus pneumoniae (serotype 1) (Sp) and PHA as a positive control. The concentrations of cytokines in resting cells were not significantly different among the three groups (data not shown). Whereas both stimuli significantly increased the levels of IL-17 and IL-22 in non-smokers and smokers, the exposure to Sp did not significantly amplify their secretion in COPD patients. The response to PHA was also partially altered in COPD patients, mainly for IL-17 and IL-22.

In order to identify the nature of the defect in COPD patients, we analyzed the intracellular cytokines in cell populations involved in the production of cytokines in response to bacteria such as NKT cells. Exposure to Sp for 24 h in non-smokers increased the percentage of IL-17⁺ and IL-22⁺ cells in NKT cells. In COPD patients, the production of both cytokines was altered in NKT cells. Concerning smokers, the IL-17 production induced by Sp was also altered in these three cell types whereas IL-22 expression was not reduced. These data showed that the response of NKT cells to infection by S. pneumoniae was altered in COPD patients. Accordingly, stimulation of NKT cells by agonists is thus interesting for the treatment of acute exacerbation of chronic obstructive pulmonary disease.

### REFERENCES

Throughout this application, various references describe the state of the art to which this invention pertains. The disclosures of these references are hereby incorporated by reference into the present disclosure.

1. A method of treating acute exacerbation of chronic obstructive pulmonary disease (COPD) in a subject in need thereof comprising administering to the subject a therapeutically effective amount of at least one natural killer T (NKT) cell agonist.

2. The method of claim 1 wherein the acute exacerbation of COPD is caused by a bacterial infection.

3. The method of claim 2 wherein the bacterial infection is due to Streptococcus pneumoniae, or Haemophilus influenzae.

4. The method of claim 1 wherein the NKT cell agonist is a alpha-galactosylceramide compound.

5. The method of claim 1 wherein the NKT cell agonist comprises a particulate entity comprising at least one alpha-galactosylceramide compound and at least one targeting agent that targets said to alpha-galactosylceramide compound to human BDCA3+ dendritic cells in vivo.

6. The method of claim 1 wherein the NKT cell agonist is an antibody

7. The method of claim 6 wherein the antibody is modified to reduce or inhibit the ability of the antibody to mediate antibody dependent cellular cytotoxicity (ADCC) and/or complement dependent cytotoxicity (CDC) functionality.

8. The method of claim 7 wherein the antibody has no Fc portion or has an Fc portion that does not bind FcyRI FcyRIII or Clq.

9. The method of claim 7 wherein the antibody has a Fc portion which is genetically or chemically altered to eliminate the Antibody dependent cell cytotoxicity (ADCC) and/or complement dependent cytotoxicity (CDC) functionality.

10. The method of claim 7 wherein the antibody comprises a heavy chain having an amino acid sequence set forth as SEQ ID NO: 1.

11. The method of claim 7 wherein the antibody comprises a light chain having an amino acid sequence set forth as SEQ ID NO: 2.

12. The method of claim 7 wherein the antibody comprises a heavy chain having an amino acid sequence set forth as SEQ ID NO: 1 and comprises a light chain having an amino acid sequence set forth as SEQ ID NO: 2.

13. The method of claim 7 wherein the antibody comprises complementarity determining regions (CDRs) of a heavy chain having an amino acid sequence set forth as SEQ ID NO: 1 and CDRs of a light chain having an amino acid sequence set forth as SEQ ID NO: 2.

14. The method of claim 1 wherein the NKT cell agonist is administered to the respiratory tract.

15. The method of claim 1 wherein the NKT cell agonist is administered to the subject in combination with one further agent selected from the group consisting of anti-bacterial agents, anti-viral agents, corticosteroids and bronchodilators.

16. The method of claim 1 wherein the NKT cell agonist is administered to the subject in combination with a vaccine which contains an antigen or antigenic composition capable of eliciting an immune response against a virus or a bacterium.

17. The method of claim 16 wherein the vaccine elicits an immune response against at least one bacterium selected from the group consisting of Streptococcus pneumoniae, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, and Bordetella pertussis.

18. The method of claim 16 wherein the vaccine is directed against Non-typeable Haemophilus influenzae (NTHi).
